(19) United States
(12) Patent Application Publication
(10) Pub. No.: US 2026/0131110 A1
(43) Pub. Date: May 14, 2026

(54) INTRAVASCULAR CATHETER WITH ADJUSTABLE STIFFNESS

(71) Applicant: KONINKLIJKE PHILIPS N.V., EINDHOVEN (NL)

(72) Inventors: Matthew SMAIL, COLORADO SPRINGS, CO (US); Ryan PEDERSEN, COLORADO SPRINGS, CO (US); Christian SANDERS, COLORADO SPRINGS, CO (US); Phil BROX, COLORADO SPRINGS, CO (US); Jacob James GELSINGER, COLORADO SPRINGS, CO (US); Robert CARVER, COLORADO SPRINGS, CO (US)

(21) Appl. No.: 19/119,138

(22) PCT Filed: Oct. 10, 2023
(86) PCT No.: PCT/EP2023/078047

§ 371 Date: Apr. 8, 2025

Related U.S. Application Data
(60) Provisional application No. 63/416,013, filed on Oct. 14, 2022.

(51) Int. Cl. A61M 25/00

(52) U.S. Cl. CPC A61M 25/0053; A61M 2025/0063; A61M 2205/0283; A61M 2205/3331; A61M 2205/502

(57) ABSTRACT

A catheter (200) includes a jacket (110), a chamber (120) and an adjustable material (230). The jacket (110) has a first stiffness and includes an outer surface (112) and an inner surface (114). The chamber (120) is enclosed within the inner surface (114). The adjustable material (230), when adjusted, provides the catheter (200) with a second stiffness which is stiffer than the first stiffness.

### BACKGROUND

Stiffness of intravascular catheters used peripherally in interventions can affect the usability of the intravascular catheter depending on the treatment area. In the treatment of peripheral vascular disease, intravascular catheters need to be flexible in order to reach distal portions of anatomy and follow the vasculature without irritating or posing a safety risk to the patient's vasculature. As catheters are designed to be more flexible, catheter stiffness may be sacrificed, and this results in less user control of the catheter tip. A more flexible catheter allows for the catheter to follow a guidewire and tortuous vasculature easily, but can be prone to buckling over longer delivery distances. As a result, more flexible catheters tend not to provide a user with adequate tip control. A stiffer catheter allows for optimal force transmission from the proximal side of the catheter to the distal tip of the catheter but may not be able to cross tortuous anatomy without posing a risk to the patient. These factors typically compete against each other.

### SUMMARY

According to an aspect of the present disclosure, a catheter includes a jacket, a chamber and an adjustable material. The jacket has a first stiffness and includes an outer surface and an inner surface. The chamber is enclosed within the inner surface. The adjustable material, when adjusted, provides the catheter with a second stiffness which is stiffer than the first stiffness.

According to another aspect of the present disclosure, a catheter includes a jacket, a chamber, a material and a controller. The jacket has a first stiffness and includes an outer surface and an inner surface. The chamber is enclosed within the inner surface. The material has an adjustable stiffness and is disposed within the chamber. When adjusted, the material provides the catheter with a second stiffness which is stiffer than the first stiffness. The controller is configured to mechanically adjust the adjustable stiffness of the material disposed within the chamber to provide the catheter with the second stiffness.

According to yet another aspect of the present disclosure, a catheter system includes a catheter and a controller. The catheter includes a jacket, a chamber, and an adjustable material. The jacket has a first stiffness and includes an outer surface and an inner surface. The chamber is enclosed within the inner surface. The adjustable material, when adjusted, provides the catheter with a second stiffness which is stiffer than the first stiffness. The controller is configured to adjust the adjustable stiffness of the adjustable material disposed within the chamber to provide the catheter with the second stiffness.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A illustrates a cross-sectional view of an intravascular catheter with adjustable stiffness, in accordance with a representative embodiment.

FIG. 1B illustrates a cross-sectional view of another intravascular catheter with adjustable stiffness, in accordance with a representative embodiment.

FIG. 2A illustrates a system with a cutout profile view of an intravascular catheter with adjustable stiffness, in accordance with a representative embodiment.

FIG. 2B illustrates another system with a cutout profile view of an intravascular catheter with adjustable stiffness, in accordance with a representative embodiment.

FIG. 2C illustrates another system with a cutout profile view of an intravascular catheter with adjustable stiffness, in accordance with a representative embodiment.

FIG. 3A illustrates a cutout profile view of an intravascular catheter with adjustable stiffness, in accordance with a representative embodiment.

FIG. 3B illustrates a cutout profile view of another intravascular catheter with adjustable stiffness, in accordance with a representative embodiment.

FIG. 4A illustrates a cutout profile view of an intravascular catheter with adjustable stiffness, in accordance with a representative embodiment.

FIG. 4B illustrates a cutout profile view of another intravascular catheter with adjustable stiffness, in accordance with a representative embodiment.

FIG. 4C illustrates another system with a cutout profile view of an intravascular catheter with adjustable stiffness, in accordance with a representative embodiment.

FIG. 5A illustrates a cross-sectional view of an intravascular catheter with adjustable stiffness, in accordance with a representative embodiment.

FIG. 5B illustrates a cross-sectional view of another intravascular catheter with adjustable stiffness, in accordance with a representative embodiment.

FIG. 6 illustrates a cross-sectional view of another intravascular catheter with adjustable stiffness, in accordance with a representative embodiment.

FIG. 7 illustrates a cross-sectional view of an intravascular catheter with adjustable stiffness, in accordance with a representative embodiment.

### DETAILED DESCRIPTION

In the following detailed description, for the purposes of explanation and not limitation, representative embodiments disclosing specific details are set forth in order to provide a thorough understanding of embodiments according to the present teachings. However, other embodiments consistent with the present disclosure that depart from specific details disclosed herein remain within the scope of the appended claims. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. Definitions and explanations for terms herein are in addition to the technical and scientific meanings of the terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below.

As described herein, an intravascular catheter may be provided with adjustable stiffness in a variety of ways. An intravascular catheter with user-controlled, variable stiffness can be flexible during delivery and stiffer when appropriate, such as when crossing tough chronic total occlusions (CTOs). An intravascular catheter that allows the user to control the stiffness profile of the catheter at different portions of treatment may optimize both trackability and deliverability of the intravascular catheter.

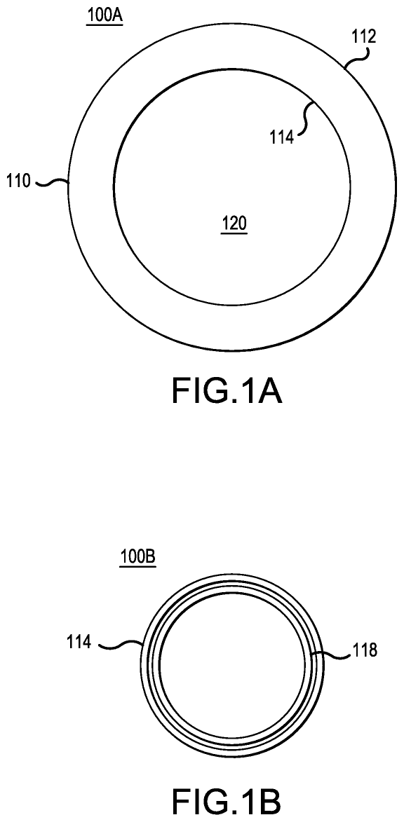

FIG. 1A illustrates a cross-sectional view of an intravascular catheter with adjustable stiffness, in accordance with a representative embodiment.

In FIG. 1A, an intravascular catheter 100A includes a jacket 110. The jacket 110 has a first stiffness and includes an outer surface 112 and an inner surface 114. A chamber 120 is enclosed within the inner surface 114. The intravascular catheter 100A includes an adjustable material which, as described herein for numerous different embodiments, provides the intravascular catheter 100A with a second stiffness which is stiffer than the first stiffness.

The material within the intravascular catheter 100A may be a material within the jacket 110 or a material within the chamber 120. In some embodiments, the material may have an ability to be linearly translated, pressurized, or electrically excited to increase stiffness of the intravascular catheter 100A. A control unit for the intravascular catheter may allow a user to manually advance, infuse, inject and/or electrically excite the material to stiffen the catheter.

The chamber 120 may be used to insert tools such as endoscopic wires and cameras, laser wires and lenses, and suction tubes into a patient. As described herein, the chamber 120 may be used to adjust stiffness of the intravascular catheter 100A using a mandrel such as a rod, or a gas or a liquid to increase pressure on the jacket 110 through the inner surface 114. A liquid may be water or saline solution. A mandrel such as a rod may comprise stainless steel., though plastic or a PTFE-based material may be used as a mandrel too.

FIG. 1B illustrates a cross-sectional view of another intravascular catheter with adjustable stiffness, in accordance with a representative embodiment.

In FIG. 1B, an intravascular catheter 100B includes the jacket with the inner surface 114, and a tube 118 disposed within the chamber and comprising the adjustable material.

In FIG. 1B, instead of a mandrel such as a rod, or a gas or liquid, the tube 118 may be used and translated just inside the inner surface 114 of the jacket 110 (not labelled in FIG. 1B). The tube 118 may be bendable, and may be a laser-cut hypo tube or a similar type of tube. The tube 118 may be tightly inserted into the chamber 120 (not labelled in FIG. 1B), and may be adjustably moved in and out of the chamber 120.

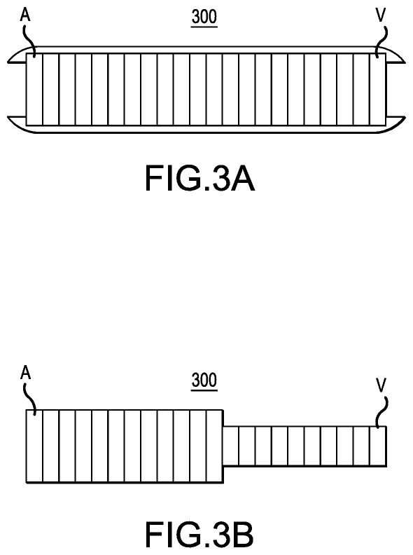

In some embodiments, instead of inserting the tube 118, the tube 118 may be fixed within the chamber 120 of the intravascular catheter 100A. In these embodiments, the tube 118 may be stimulated, such by an electrical signal, to stiffen and thereby stiffen the intravascular catheter 100B. In some embodiments based on FIG. 1B and FIG. 3A and FIG. 3B, one or more selected subsection(s) of the tube 118 may be selectively stimulated to stiffen without stiffening other subsections on one or even both sides of the subsection(s) being stiffened. In this way,

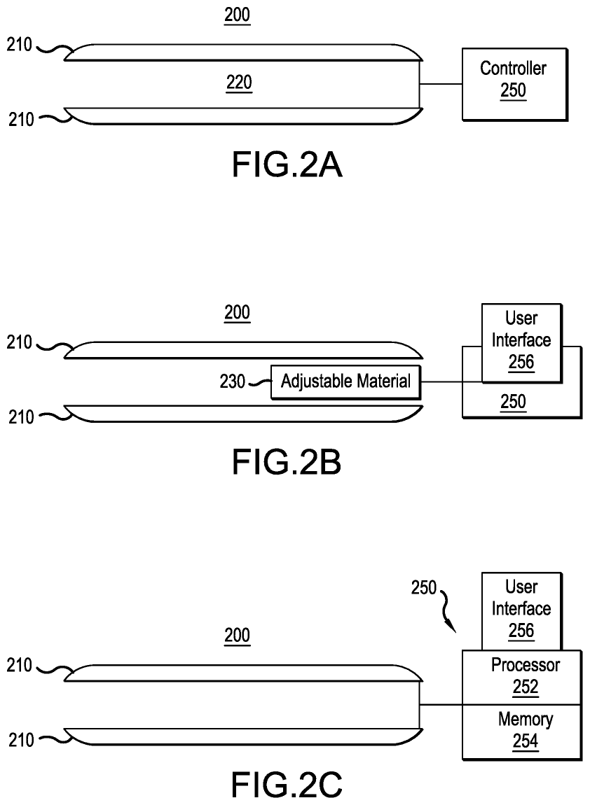

FIG. 2A illustrates a system with a cutout profile view of an intravascular catheter with adjustable stiffness, in accordance with a representative embodiment.

In FIG. 2A, a catheter 200 with a jacket 210 around a chamber 220 is controlled by a controller 250. The controller 250 is configured to adjust the adjustable material to provide the catheter 200 with a second stiffness.

FIG. 2B illustrates another system with a cutout profile view of an intravascular catheter with adjustable stiffness, in accordance with a representative embodiment.

In FIG. 2B, the catheter 200 with the jacket 210 around the chamber 220 is controlled by a controller 250, and the controller 250 is provided with a user interface 256. The controller 250 may be configured to adjust the adjustable material to provide the catheter 200 with the second stiffness. In FIG. 2B, the adjustable material 230 may be a mandrel such as a rod configured to be linearly translated within the chamber of the catheter 200. The user interface 256 is configured to allow a user to adjust the adjustable material. In FIG. 2B, the user interface 256 may be a mechanical interface, an electronic interface, or both. For example, the user interface 256 may be an electronic interface that allows a user to enter a number or set of numbers to specify subsections of the catheter for which additional stiffness should be generated. Alternatively, the user interface 256 may be a mechanical interface that allows a user to linearly translate an adjustable material disposed within the chamber.

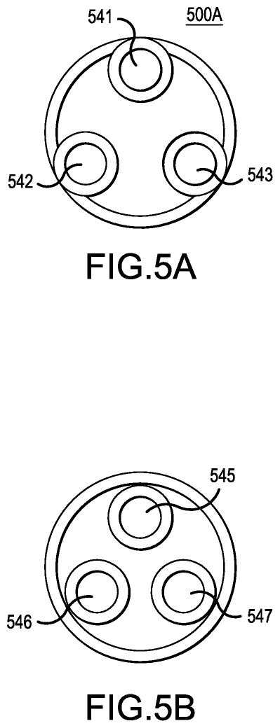

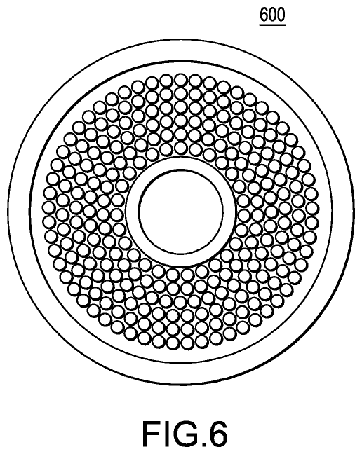

In some embodiments, the chamber of the catheter 200 in FIG. 2B may include at least one channel comprising the adjustable material. The adjustable material may comprise a stiffening agent configured to be linearly translated within the at least one channel. Examples of embodiments with multiple discrete channels within a chamber of catheter are shown in FIG. 5A, FIG. 5B and FIG. 6.

FIG. 2C illustrates another system with a cutout profile view of an intravascular catheter with adjustable stiffness, in accordance with a representative embodiment.

In FIG. 2C, the catheter 200 with the jacket 210 around the chamber 220 is controlled by a controller 250, and the controller 250 has a processor 252, a memory 254, and the user interface 256. The controller 250 may be a digital controller. The controller 250 in FIG. 2C is configured to adjust the adjustable material to provide the catheter 200 with the second stiffness. The user interface is configured to allow a user to adjust the adjustable material.

In some embodiments, the adjustable material may be an ionomer plastic, and the user interface 256 may be configured to electrically excite the ionomer plastic to stiffen. In some embodiments, the jacket 210 may comprise the ionomer plastic as the adjustable material.

The memory 254 stores instructions and the processor 252 executes the instructions. The controller 250 may also include additional interfaces other than the user interface 256, such as a second interface, a third interface, and a fourth interface. One or more of the interfaces may include ports, disk drives, wireless antennas, or other types of receiver circuitry that connect the controller 250 to other electronic elements.

The user interface 256 may comprise one or more buttons or keys, a mouse, a microphone, a speaker, or other elements that users can use to interact with the controller 250 such as to enter instructions and receive output.

The processor 252 may be considered a representative example of a processor of a controller and executes instructions to implement some or all aspects of methods and processes described herein. The processor 252 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 252 is an article of manufacture and/or a machine component. The processor 252 is configured to execute software instructions to perform functions as described in the various embodiments herein. The processor 252 may be a general-purpose processor or may be part of an application specific integrated circuit (ASIC). The processor 252 may also be a microprocessor, a microcomputer, a processor chip, a controller, a microcontroller, a digital signal processor (DSP), a state machine, or a programmable logic device. The processor 252 may also be a logical circuit, including a programmable gate array (PGA), such as a field programmable gate array (FPGA), or another type of circuit that includes discrete gate and/or transistor logic. The processor 252 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The memory 254 may communicate with the processor 252 via a bus (not shown). The memory 254 stores instructions used to implement some or all aspects of methods and processes attributed to the controller 250 in FIG. 2C. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The memory 254 is an articles of manufacture and/or a machine component. The memory 254 is a computer-readable mediums from which data and executable software instructions can be read by a computer (e.g., the processor 252). The memory 254 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

In an embodiment, dedicated hardware implementations, such as application-specific integrated circuits (ASICs), field programmable gate arrays (FPGAs), programmable logic arrays and other hardware components, are constructed to methods attributed to the controller 250. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In the embodiments of FIG. 2A, FIG. 2B and FIG. 2C, the jacket 210 of the catheter 200 may be electrically excitable. For example, the jacket 210 may comprise ionomer plastics or nitinol as the adjustable material. The electrically excitable adjustable material may be used in the working length of the jacket 210 of the catheter 200 and may be electrically excited in order to change the stiffness profile of the catheter 200. An ionomer may be used in the material of the jacket 210 or as a mandrel. Similarly, nitinol may be used as a mandrel. When used as a mandrel, the nitinol or ionomer is not physically translated to stiffen the catheter 200, and instead the nitinol or ionomer is electrically excited to stiffen the catheter 200.

FIG. 3A illustrates a cutout profile view of an intravascular catheter with adjustable stiffness, in accordance with a representative embodiment.

In FIG. 3A, an intravascular catheter 300 is divided into portioned subsections. The intravascular catheter 300 is provided with an adjustable stiffness which is adjustable at a plurality of discrete subsections along the intravascular catheter 300 The intravascular catheter 300 may have an adjustable stiffness profile along the catheter, whether from the distal end of the intravascular catheter 300, from the proximal end of the intravascular catheter 300, or for one or more discrete subsections between the distal end and the proximal end of the intravascular catheter 300.

FIG. 3B illustrates a cutout profile view of another intravascular catheter with adjustable stiffness, in accordance with a representative embodiment.

In FIG. 3B, the intravascular catheter 300 is again divided into partitioned subsections, and is again provided with an adjustable stiffness which is adjustable at a plurality of discrete subsections along the catheter. As shown, the partitioned subsections on the left are larger than the partitioned subsections on the right. The larger portioned subsections on the left may have a larger diameter, such as due to inflation of a gas or liquid that is separably provided in one or a group of subsections.

The partitioned subsections in FIG. 3A and FIG. 3B may be applicable to multiple different embodiments herein. For example, the partitioned subsections may be used for embodiments in which adjustable stiffness may be selectively provided, and this may include adjustable stiffness provided selectively by electrical stimulation or by pressurized gas or liquid. Selective stiffness may be provided at a granularity on the scale of 10 centimeters or more, though granularity may be improved when catheters are provided with improved discreteness in subsections. A controller described herein, such as the controller 250 in FIG. 2C, may control stiffness using specific coordinates corresponding to individual subsections shown in FIG. 3A and FIG. 3B. The adjustable stiffness may be selectively provided in an inner section of the intravascular catheter 300, between the distal end and the proximal end and without necessarily including a subsection at the distal end or a subsection at the proximal end.

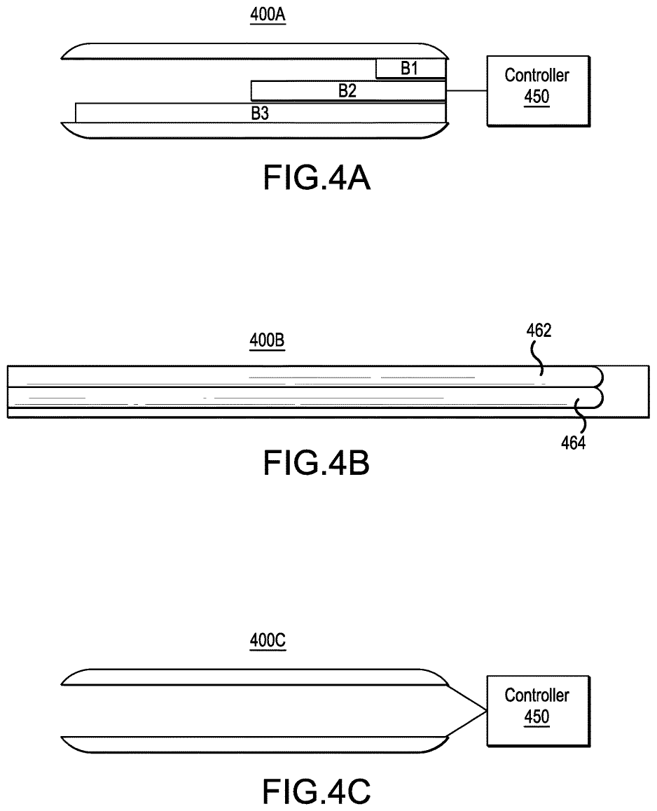

FIG. 4A illustrates a cutout profile view of an intravascular catheter with adjustable stiffness, in accordance with a representative embodiment.

In FIG. 4A, an intravascular catheter 400A is provided with a controller 450. Three slidable elements B1, B2 and B3 are configured to slide into the chamber of the intravascular catheter 400A. The controller 450 is configured to adjust the adjustable material to provide the intravascular catheter 400A with the second stiffness. In FIG. 4A, the adjustable material includes three insertion portions including a first insertion portion B1, a second insertion portion B2 and a third insertion portion B3. The three insertion portions may be configured to be linearly driven into the chamber of the intravascular catheter 400A. As shown, the three insertion portions may be driven to different depths of the intravascular catheter 400A. While three insertion portions are shown in FIG. 4A, the number of insertion portions may be more or less than three in other embodiments. Additionally, while all three insertion portions are shown aligned at the proximal end of the intravascular catheter 400A in FIG. 4A, one or more of the three insertion portions may be controlled to move further into the chamber and beyond the proximal end.

In some embodiments, each of the three insertion portions may comprise a stiffening agent which is configured to be linearly translated with one or more channels.

In some embodiments, one or more insertion portions may be provided as tubes such as the tube 118 in FIG. 1B, and another of the insertion portions may be driven through the insertion portions serving as tubes to stiffen only one or more selected subsections of the intravascular catheter 400A. In such embodiments, the adjustable material may be linearly translated within the intravascular catheter 400A under the control of the controller 450.

In some embodiments, the insertion portions driven into the chamber may have a first stiffness when driven, and may be selectively stiffened once in the proper position. For example, insertion portions driven into the chamber to be selectively stiffened may be selectively stiffened by gas or liquid pressure or may be electrically excited. As an example, water or a saline solution may be used as a liquid stiffening agent. The insertion portions may be positioned first, and then stiffened to provide the intravascular catheter 400A with the second stiffness. In some embodiments, one or more of the insertion portions may comprise a stent disposed within the chamber, and the stent may be selectively expanded to stiffen the chamber and thereby stiffen the intravascular catheter 400A.

FIG. 4B illustrates a cutout profile view of another intravascular catheter with adjustable stiffness, in accordance with a representative embodiment.

In FIG. 4B, the intravascular catheter 400B is provided with a first balloon 462 and a second balloon 464. Although two balloons are shown in FIG. 4B, an intravascular catheter 400B may include one balloon or more than two balloons without departing from the spirit and scope of the teachings herein. The balloons may be inflated with air or a solution such as saline or contras. In most embodiments using balloons, the balloons will be compliant balloons which inflate only to a maximum diameter, though non-compliant balloons may be used in limited circumstances so long as proper safety measures are in place.

Although FIG. 4B does not show a controller, embodiments based on FIG. 4B may include a controller such as a user interface that allows a user to selectively position and inflate the first balloon 462 and the second balloon 464. A user interface of such a controller may be used to push and pressurize the fluid media in the first balloon 462 and the second balloon 464.

In the embodiment of FIG. 4B, the first balloon 462 and the second balloon 464 may be bladders that can be inflated and deflated with a fluid media to increase the stiffness. In FIG. 4B, the first balloon 462 and the second balloon 464 extend almost throughout the intravascular catheter 400B, from the distal portion of the intravascular catheter 400B almost to the proximate end of the intravascular catheter 400B. The first balloon 462 and the second balloon 464 may be permanently or temporarily inserted into the chamber of the intravascular catheter 400B, and selectively inflated and then collapsed.

In some embodiments alternative to the embodiment of FIG. 4B, the interstitial space inside the jacket of the intravascular catheter 400B and around catheter components may be used as a bladder, with fluid being infused into that space rather than into a designated bladder, such that the space between fibers and inner lumen is used as the inflation bladder. An embodiment showing with this this configuration is shown in FIG. 6, though one or more of the fibers shown in FIG. 6 may alternatively comprise balloon(s) as in FIG. 4B.

FIG. 4C illustrates another system with a cutout profile view of an intravascular catheter with adjustable stiffness, in accordance with a representative embodiment.

In FIG. 4C, the intravascular catheter 400C is provided with a controller 450. The controller 450 is configured to adjust the adjustable material to provide the intravascular catheter 400C with a second stiffness which is stiffer than the first stiffness. The controller 450 may comprise memory that stores instructions and processor that executes the instructions, and physical user interfaces that allow a user to enter instructions or manipulate a button, switch or knob to allow the user to adjust the adjustable material.

In the embodiment of FIG. 4C, the jacket of the intravascular catheter 400C may comprise the adjustable material, and the controller 450 may be configured to electrically excite the adjustable material. For example, the jacket may comprise an ionomer plastic which is configured, when electrically excited, to stiffen.

Alternatively, in FIG. 4C, a woven material may be housed within the chamber and may include a distal end fixed relative to the jacket and a proximal end configured to be linearly advanced relative to the distal end. The controller 450 may be configured to physically push the woven material from the proximal end, under the control of a user.

FIG. 5A illustrates a cross-sectional view of an intravascular catheter with adjustable stiffness, in accordance with a representative embodiment.

In FIG. 5A, the catheter 500A includes a first channel 541, a second channel 542 and a third channel 543.

In the embodiment of FIG. 5A a material may be linearly translated through the first channel 541, the second channel 542 and/or the third channel 543. As an example, the material may be a stiffening agent such as a mandrel that can be linearly translated within the first channel 541, the second channel 542 and/or the third channel 543. Each of the first channel 541, the second channel 542 and/or the third channel 543 in the embodiment of FIG. 5A may consist of a multi-lumen jacket material.

In other embodiments, separate balloons may be positioned within and inflated within any or all of the first channel 541, the second channel 542 and/or the third channel 543.

FIG. 5B illustrates a cross-sectional view of another intravascular catheter with adjustable stiffness, in accordance with a representative embodiment.

In FIG. 5B, the catheter 500B includes a first channel 545, a second channel 5456 and a third channel 547. In the embodiment of FIG. 5B a material may again be linearly translated through the first channel 545, the second channel 546 and/or the third channel 547. The material may again be a stiffening agent such as a mandrel that can be linearly translated within the first channel 545, the second channel 546 and/or the third channel 547. In FIG. 5B, each of the first channel 545, the second channel 546 and/or the third channel 547 may consist of an inner lumens separate and apart from the jacket.

In other embodiments, separate balloons may be positioned within and inflated within any or all of the first channel 545, the second channel 546 and/or the third channel 547.

FIG. 6 illustrates a cross-sectional view of another intravascular catheter with adjustable stiffness, in accordance with a representative embodiment.

In FIG. 6, the intravascular catheter 600 is substantially filled with channels. The interstitial space inside the jacket and around the channels with the catheter components may be used as a bladder. Fluid may be infused into the interstitial space rather than into a designated bladder. As an example, the channels in FIG. 6 may be fibers for endoscopes, lasers, suction tubes, and other types of elements provided as components in catheters. The chamber may be configured to be filled with a fluid around the components of the catheter disposed within the chamber.

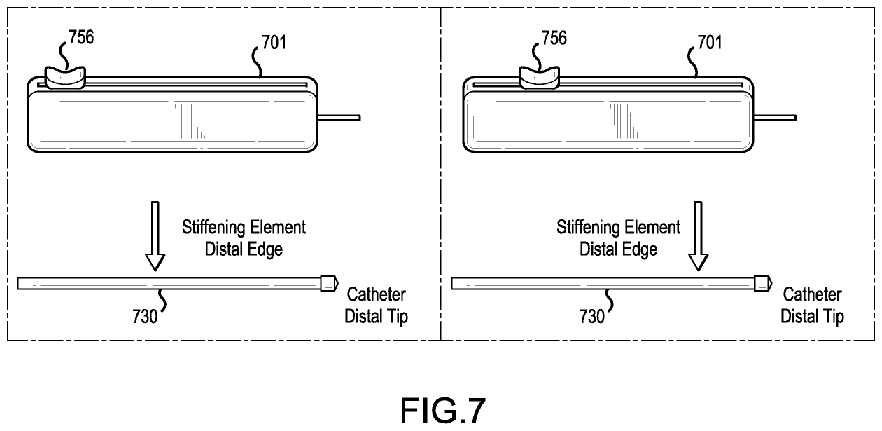

FIG. 7 illustrates a cross-sectional view of an intravascular catheter with adjustable stiffness, in accordance with a representative embodiment.

In FIG. 7, a slidable button 756 is provided on the top of an adjustment system 701. The slidable button 756 is used to translate motion to slide the stiffening element 730 to an intravascular catheter. A proximal end of the catheter is provided with stiffening element 730.

On the left side of FIG. 7, the stiffening element may be a stiffening agent in a proximal-most position, such that the catheter has the longest flexible length. On the right side of FIG. 7, the slidable button 756 is slid forward, moving the stiffening element 730 distally inside the working length of the catheter. This shortens the flexible length of the catheter and stiffens the proximal portion of the catheter.

The adjustment system 701 provides the slidable button 756 as a user interface that allows the user to control the position of the stiffening element 730 as a mandrel along the length of the catheter. As the user advances the slidable button 756 as the user interface on the adjustment system 701, the mandrel as the stiffening element 730 advances distally into the working length of the catheter. The slidable button 756 may be provided with small ridges or another form of frictional surface on the top, to enhance the ability of a user to move the stiffening element 730. Additionally, physical markers may be provided along a channel through which the slidable button 756 slides, such as every 1 centimeter,

On the left side in FIG. 7, the button and the stiffening agent are in the proximal-most position, meaning the catheter has the longest flexible length. On the right side in FIG. 7, the button is slid forward and moves the stiffening element distally inside the working length of the catheter. This shortens the flexible length of the catheter and stiffens the proximal portion of the catheter.

Accordingly, intravascular catheter with adjustable stiffness enables users to vary the stiffness in intravascular catheters, including at specific portions of the intravascular catheters. The intravascular catheter with user-controlled and variable stiffness may be made flexible during delivery and stiffer when appropriate. Intravascular catheters described herein may allow users to control the stiffness profile of the intravascular catheter at different portions of treatment, so as to optimize both trackability and deliverability of the intravascular catheter.

Although intravascular catheter with adjustable stiffness has been described with reference to several exemplary embodiments, it is understood that the words that have been used are words of description and illustration, rather than words of limitation. Changes may be made within the purview of the appended claims, as presently stated and as amended, without departing from the scope and spirit of intravascular catheter with adjustable stiffness in its aspects. For example, in some other example embodiments, a stiffener outer diameter may be changed. As the outer diameter of the materials in the cross-section of a catheter is increased, so is the second moment area of the catheter, and this creates a stiffer catheter that is more difficult to bend. In some example embodiments, a self-expanding and self-collapsing stent-like structure may be provided inside the outer jacket of the catheter and may be expanded when additional stiffness is desired and collapsed when the added stiffness is no longer required. Expansion of the structure may be provided due to linear motion of a containing material, such as if the stent structure is of a self-expanding stent, or the stent may be electrically excited to increase the outer diameter of the stent. In some other example embodiments, a woven structure such as a finger trap may be housed within the catheter outer jacket. With the distal end of the woven structure fixed in place, the proximal side of the structure may be linearly advanced distally, increasing the outer diameter of the woven structure.

Although intravascular catheter with adjustable stiffness has been described with reference to particular means, materials and embodiments, intravascular catheter with adjustable stiffness is not intended to be limited to the particulars disclosed; rather intravascular catheter with adjustable stiffness extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A catheter, comprising:
a jacket with a first stiffness and comprising an outer surface and an inner surface; and
a chamber enclosed within the inner surface, and
an adjustable material that, when adjusted, provides the catheter with a second stiffness which is stiffer than the first stiffness.

2. The catheter of claim 1, further comprising:
a controller configured to adjust the adjustable material to provide the catheter with the second stiffness.

3. The catheter of claim 2, wherein the controller comprises a user interface configured to allow a user to adjust the adjustable material.

4. The catheter of claim 3, wherein the user interface comprises a slidable button connected to the adjustable material and configured to linearly advance the adjustable material into the chamber.

5. The catheter of claim 1, wherein the first stiffness is adjustable to the second stiffness at a plurality of discrete subsections along the catheter.

6. The catheter of claim 1, wherein the catheter has an adjustable stiffness profile along the catheter.

7. The catheter of claim 2, wherein the adjustable material is disposed within the chamber and the controller is configured to linearly translate the adjustable material within the chamber.

8. The catheter of claim 2, wherein the adjustable material is disposed within the chamber and the controller is configured to pressurize the adjustable material within the chamber.

9. The catheter of claim 2, wherein the jacket comprises the adjustable material and the controller is configured to electrically excite the adjustable material.

10. The catheter of claim 1, further comprising:
at least one channel within the chamber and comprising the adjustable material, wherein the adjustable material comprises a stiffening agent configured to be linearly translated within the at least one channel.

11. The catheter of claim 1, further comprising:
a tube disposed within the chamber and comprising the adjustable material.

12. The catheter of claim 1, further comprising:
at least one balloon disposed within the chamber, configured to be selectively inflated, and comprising the adjustable material.

13. The catheter of claim 1, wherein the chamber is configured to be filled with a fluid around at least one component of the catheter disposed within the chamber.

14. The catheter of claim 1, further comprising:
an ionomer plastic comprising the adjustable material and configured, when electrically excited, to stiffen.

15. The catheter of claim 1, wherein the jacket comprises an ionomer plastic.

16. The catheter of claim 1, further comprising:
a stent comprising the adjustable material and disposed within the chamber, wherein the stent is configured to be selectively expanded to stiffen the chamber.

17. The catheter of claim 1, further comprising:
a woven structure housed within the chamber and comprising a distal end fixed relative to the jacket and a proximal end configured to be linearly advanced relative to the distal end.

18. A catheter, comprising:
a jacket with a first stiffness and comprising an outer surface and an inner surface; and
a chamber enclosed within the inner surface;
a material with an adjustable stiffness and disposed within the chamber that, when adjusted, provides the catheter with a second stiffness which is stiffer than the first stiffness, and
a controller configured to mechanically adjust the adjustable stiffness of the material disposed within the chamber to provide the catheter with the second stiffness.

19. A catheter system, comprising:
a catheter, comprising a jacket with a first stiffness and comprising an outer surface and an inner surface, a chamber enclosed within the inner surface, and an adjustable material that, when adjusted, provides the catheter with a second stiffness which is stiffer than the first stiffness; and
a controller configured to adjust the first stiffness to the second stiffness of the adjustable material disposed within the chamber to provide the catheter with the second stiffness.
